(19) United States
(12) Patent Application Publication
(10) Pub. No.: US 2004/0205831 A1
(43) Pub. Date: Oct. 14, 2004

(54) Transgenic SHIP2 animals and in vivo screening methods

(76) Inventors: Mark W. Sleeman, Mahopac, NY (US); Katherine E. Wortley, Briarcliff Manor, NY (US)

Correspondence Address: REGENERON PHARMACEUTICALS, INC, 777 OLD SAW MILL RIVER ROAD, TARRYTOWN, NY, 10591, US

(21) Appl. No.: 10/411,187

(22) Filed: Apr. 10, 2003

(51) Int. Cl. A01K 67/027

(52) U.S. Cl. 800/3; 800/14; 800/18

(57) ABSTRACT

Transgenic animals comprising an alteration in the endogenous SH2 domain containing inositol 5-phosphatase (SHIP2), including both knock-in, knockouts, and knock-in/knockouts are described, as well as in vivo methods for identifying inhibitors of SHIP2. Such inhibitors are potential therapeutics for treatment of obesity and/or glucose intolerance resulting from a high fat diet.

### BACKGROUND OF THE INVENTION

1. Field of the Invention

This invention is related to non-human animals having a deletion of the human SH2 domain containing inositol 5-phosphatase (SHIP2) gene; methods for generating SHIP2 knockout animals; and methods for using SHIP2 knockout animals to identify agents capable of inhibiting human SHIP2 protein, as well as therapeutic uses for such identified molecules.

2. Description of Related Art

The phosphatidylinositol 3-kinase (PI3K)-Akt pathway is an evolutionally conserved signaling cassette that functions in mammals to transduce survival signals in response to growth factor stimulation. The binding of growth factors to their cognate tyrosine receptors on the cell surface leads to the intracellular recruitment and activation of PI3-K, resulting in the generation of phosphatidylinositol 3,4-bisphosphate (PtdIns(3,4)P₂) (or PIP₂) and phosphatidylinositol 3,4,5-triphosphate (PtdIns(3,4,5)P₃) (or PIP₃). These phosphorylated lipids are able recruit the serine-threonine kinase Akt to the plasma membrane, where it becomes fully active, as well as act as second messengers in a number of diverse biological processes, such as mitogenesis, oncogenic transformation, apoptosis, and membrane trafficking agents. The cellular content and localization of PIP₂ and PIP₃ is highly regulated process and one well characterized mechanism is through their hydrolysis by a family of inositol polyphosphate 5-phosphatases.

SH2 domain containing inositol 5-phosphatase 1 (SHIP1), originally identified as a Shc binding protein and having 5′ phosphatase activity, was initially proposed as the main negative regulator of insulin signaling. Given the restricted expression of SHIP-1 to hematopoietic cells however, it was suggested that the more broadly expressed isozyme, SHIP2, may be more intimately involved in signaling from receptor tyrosine kinases in insulin sensitive tissues. Subsequent experiments showed that decreased expression or deficiency of SHIP2 lead to increased insulin sensitivity, and that overexpression of SHIP2 in various insulin sensitive cells lines lead to decreased insulin signaling.

U.S. Pat. No. 6,025,198 (Bennett et al.) describes SHIP2 antisense molecules and an in vitro method of inhibiting SHIP2 expression in human cells/tissues with the SHIP2 antisense molecules disclosed.

### SUMMARY OF THE INVENTION

The function of SH2 domain containing inositol 5-phosphatase 2 (SHIP2) in obesity and metabolism was elucidated in the experiments described below with the use of a SHIP2 knockout animal exhibiting resistance to the development of obesity when placed on a high fat diet.

Accordingly, in a first aspect, the invention features a non-human transgenic animal comprising an alteration in the endogenous SHIP2 gene. In one embodiment, the alteration in the endogenous SHIP2 gene is a disruption of one or both SHIP2 alleles, e.g., a heterozygous or homozygous SHIP2 knockout animal. In a more specific embodiment, the SHIP2 knockout animal is homozygous for the disrupted SHIP2 gene, and exhibits resistance to development of obesity when placed on a high fat diet.

In another embodiment, the alteration is a replacement of the endogenous SHIP2 gene with a human SHIP2 gene, e.g., a SHIP2 knock-in animal. Preferably, the SHIP2 knock-in animal is homozygous for the human SHIP2 gene.

The SHIP2 transgenic animals of the invention are useful in a variety of ways. The SHIP2 knockout is useful as an animal model of SHIP2-mediated conditions. The SHIP2 knock-in is useful for identifying agents capable of inhibiting SHIP2 activity in vivo. Agents identified by the in vivo screening method of the invention are potential therapeutic agents for the treatment of human obesity. More generally, such agents may be useful for treatment of SHIP2-related conditions, including weight gain resulting from a high fat diet, insensitivity to insulin caused by obesity or consumption of a high fat diet, and/or a slow or depressed metabolism.

In a second aspect, the invention features an in vivo screening method for identifying agents capable of inhibiting SHIP2, comprising (a) administering a test agent to SHIP2 knock-in animal; and (b) determining the ability of the test agent to inhibit SHIP2. The term “inhibition of SHIP2” includes inhibition of SHIP2 protein function (activity), as well as inhibition of SHIP2 gene expression. The ability of a test agent to inhibit SHIP2 relative to a control may be determined in a number of ways, including directly or indirectly. Direct measurement of SHIP2 activity includes, for example, measuring phosphatase activity or Akt activation. Direct measurement of SHIP2 gene expression includes, for example, measuring SHIP2 mRNA. Indirect measurements of SHIP2 activity or expression include, for example, determining weight gain in response to a high fat diet relative to a control animal, wherein a decreased weight gain relative to the control animal indicates inhibition of SHIP2 activity and/or expression.

In a third aspect, the invention features a therapeutic method for treating obesity, inducing weight loss, increasing metabolism, and/or decreasing or preventing the obesity associated insulin resistance, comprising administering a therapeutically effective amount of an agent capable of inhibiting SHIP2-mediated activity or expression. In specific embodiments, the agent capable of inhibiting SHIP2 is a nucleic acid which is an SHIP2 antisense molecule, a ribozyme or triple helix, or a short interfering RNA (siRNA) capable of silencing SHIP2 gene expression. In another embodiment, the agent is an inhibitor of SHIP2 identified by the in vivo screening assay of the invention. In specific embodiments, the SHIP2 inhibitor is an antagonist of SHIP2, such as an cell permeable peptide which binds the SH2 domain of SHIP2. More specifically, the SHIP2 inhibitor may be a cell permeable antibody specific to SHIP2 or to an activator of SHIP2. The antibody may be polyclonal, monoclonal, chimeric, humanized, or a wholly human antibody. In another embodiment, the therapeutic method of the invention comprising administering an agent of the invention with a second agent. In this embodiment, the therapeutic method may allow a decreased amount of the second agent to be administered when administered in combination with an agent of the invention.

In a fourth aspect, the invention features pharmaceutical compositions comprising a SHIP2 inhibitor useful for treatment of obesity, inducing weight loss, increasing metabolism and/or decreasing or preventing insulin resistance. In one embodiment, an agent identified by a screening method of the invention. In another embodiment, the agent is a SHIP2 antisense molecule.

Other objects and advantages will become apparent from a review of the ensuing detailed description.

### BRIEF DESCRIPTION OF THE FIGURES

FIG. 1: Lac-Z expression in the brain of SHIP2^{-/-} knockout mice.

FIG. 2: LacZ expression in the (A) in the abdominal wall; (B) heart, (C) hind limb of SHIP2^{-/-} knockout mice.

FIG. 3: Analysis of body weight response of SHIP2^{-/-} knockout mice to high fat diet. Wild-type and SHIP2^{-/-} mice of 8-10 weeks of age that have been maintained on normal chow were placed on a high fat diet (45% fat; Research Lab, N.J.) and their bodyweight measured at the same time each week for 6 weeks. Body weight gain is expressed as (A) a percentage of starting body weight, and (B) absolute body weight (grams). Each data point represents the mean±SEM of n 6-8 animals.

FIG. 4: Analysis of body composition by pDEXA of SHIP2^{-/-} knockout mice after a high fat diet. Wild-type and SHIP-2^{-/-} mice were assessed before (8-10 weeks of age; solid bars) and after 6 weeks of a high fat diet (14-16 weeks of age; open Bars) by dual emission X ray absorption (pDEXA). Data is expressed as the mean±SEM of n=6-8 animals for the derived parameters of (A) bone mineral density (grams); (B) bone mineral content; (C) absolute lean mass (grams); (D) absolute fat mass (grams); (E) percentage lean mass (% of total bodyweight); and (F) percentage fat mass (% of total body weight).

FIG. 5: Analysis of glucose homeostasis of SHIP2^{-/-} knockout mice after a high fat diet. Wild-type and SHIP2^{-/-} mice of 8-10 weeks of age that have been maintained on normal chow or fed a high fat diet for 6 weeks were (A) fasted for 16-18 hrs before being administered a bolus of glucose and blood glucose assessed at 30, 60 and 120 minutes as part of the standard Oral glucose tolerance test; or (B) were allowed free access to food before administration of a bolus of humans Insulin (1 unit/kg) and assessed at 30, 60 and 120 minutes for blood glucose as part of the standard insulin tolerance test.

FIG. 6: Analysis of metabolic parameters of SHIP2^{-/-} knockout mice after a high fat diet. Wild-type and SHIP2^{-/-} mice of 14-16 weeks of age that have been maintained on the high fat diet for 6 weeks were acclimated for a 24 hr period in metabolic cage and ad lib food intake, O₂ consumption, CO₂ production and activity continuously monitored for the next 48 hr by indirect calorimetry. For all data each interval represents approximately 1 hr with the black bar indicating the dark period. The data point represents the mean±SEM of n=6-8 animals. (A) cumulative food intake over the 72 hour period (grams); (B) food eaten per feeding bout (grams); and (C) basal metabolic rate (ml/kg/hr), which represents the average V0₂ during the light periods.

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a method” include one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to describe the methods and/or materials in connection with which the publications are cited.

Definitions

By the term “inhibitor” is meant a substance which retards or prevents a chemical or physiological reaction or response. A “SHIP2 inhibitor” includes an agent capable of inhibiting SHIP2 activity or function, as well as agents capable of inhibiting or interfering with SHIP2 gene expression. Examples of inhibitors include, but are not limited to, antisense molecules, siRNA molecules, antibodies, antagonists and their derivatives.

A “SHIP2-mediated condition” means an undesirable condition associated with SHIP2 function or expression. For example, the SHIP2 knockout animals of the invention demonstrate an improved resistance to obesity when placed on a high fat diet, relative to wild-type (control) animals.

A “knock-out” animal is an animal generated from a mammalian cell that carries a genetic modification resulting from the insertion of a DNA construct targeted to a predetermined, specific chromosomal location that alters the function and/or expression of a gene that was at the site of the targeted chromosomal location. A transgenic “knock-in” animal is an animal generated from a mammalian cell that carries a genetic modification resulting from the insertion of a DNA construct targeted to a predetermined, specific chromosomal location wherein the inserted DNA provides an additional function and/or replaces the function of the gene that was at the site of the targeted chromosomal location. A transgenic “knock-in” may also alter the function and/or expression of a gene that was at the site of the targeted chromosomal location. In both cases, the DNA construct may encode a reporter protein such as lacZ, protein tags, and proteins, including recombinases such as Cre and FLP.

General Description

The experiments described below identify the function of SH2 domain containing inositol 5-phosphatase 2 (SHIP2) as involved in metabolic processes, for example those involved in weight regulation, as well as insulin sensitivity. Accordingly, these discoveries provide new methods for the treatment of SHIP2-mediated conditions, such as obesity, and associated depressed metabolic activity and insulin resistance. Further, the invention provides screening assays for identification of molecules capable of inhibiting SHIP2-mediated activity in vivo.

Screening Assays

The present invention provides in vivo methods for identifying agents (e.g., candidate compounds or test compounds) that are capable of inhibiting human SH2 domain containing inositol 5-phosphatase 2 (SHIP2)-mediated activity in a non-human transgenic animal model. Preferably, the invention provides methods for identifying agents capable of treating or preventing obesity and insulin resistance. Agents identified through the screening method of the invention are potential therapeutics for use in treating a subject in need thereof.

Examples of suitable animals for use as a non-human transgenic animal model include, but are not limited to, mice, rats, rabbits, monkeys, guinea pigs, dogs and cats. In a preferred embodiment, the transgenic animal model is a homozygous knock-in of the human SHIP2 gene. In accordance with this embodiment, the test compound or a control compound is administered (e.g., orally, rectally or parenterally such as intraperitoneally or intravenously) to a suitable animal and the effect on the SHIP2-mediated activity is determined. More specifically, this method may be used to identify an agent capable of inhibiting weight gain, enhancing weight loss, decreasing or preventing insulin resistance or enhancing metabolism.

Examples of agents include, but are not limited to, nucleic acids (e.g., DNA and RNA), carbohydrates, lipids, proteins, peptides, peptidomimetics, small molecules and other drugs. Agents can be obtained using any of the numerous approaches in combinatorial library methods known in the art. Test compounds further include, for example, antibodies (e.g., polyclonal, monoclonal, humanized, anti-idiotypic, chimeric, and single chain antibodies as well as Fab, F(ab′).sub.2, Fab expression library fragments, and epitope-binding fragments of antibodies). Further, agents or libraries of compounds may be presented, for example, in solution, on beads, chips, bacteria, spores, plasmids or phage.

The in vivo screening method of the invention allows identification of agents capable of inhibiting SHIP2 activity, including SHIP2 function and/or SHIP2 expression. Determination of the ability of a test agent to inhibit SHIP2 activity includes both direct and indirect determinations. For example, the ability of a test agent to inhibit SHIP2 activity may be determined by administering the test agent to a model animal expressing the human SHIP2 protein, obtaining a biological sample, and determining the SHIP2 function and/or activity relative to a control by, for example, quantification of the corresponding SHIP2 mRNA, and/or measuring the activation of the Akt pathway. Methods for determining inhibition of SHIP2 are described in co-pending U.S. Ser. No. 10,086,201 filed 28 Feb. 2002, herein specifically incorporated by reference in its entirety. Biological samples may include biological fluids, such as blood or serum, cells or tissues, such as muscle biopsies, etc. Indirect methods for determining the ability of a test agent to inhibit SHIP2 activity may be determined by, for example, by measuring weight gain relative to a wild-type control animal in response to a high fat diet Indirect methods further include improved hyperglycemia and/or glucose tolerance, or a higher basal metabolic rate relative to the control group. A combination of these parameters may also be used to identify agents capable of inhibiting SHIP2 activity in vivo.

Antibodies and Inhibitory Peptides to Human SHIP2 Protein

According to the invention, a human SHIP2 protein, protein fragment, derivative or variant, may be used as an immunogen to generate immunospecific antibodies. Further, the present invention includes antibodies to compounds capable of binding SHIP2. Such immunogens can be isolated by any convenient means known to the art. Antibodies of the invention include, but are not limited to polyclonal, monoclonal, bispecific, humanized or chimeric antibodies, single chain antibodies, Fab fragments and F(ab′) fragments, fragments produced by a Fab expression library, anti-idiotypic (anti-Id) antibodies, and epitope-binding fragments of any of the above. The term “antibody” as used herein refers to immunoglobulin molecules and immunologically active portions of immunoglobulin molecules, i.e., molecules that contain an antigen binding site that specifically binds an antigen. The immunoglobulin molecules of the invention can be of any class (e.g., IgG, IgE, IgM, IgD and IgA) or subclass of immunoglobulin molecule. The present invention provides for an antibody which specifically binds human SHIP2 and is useful to inhibit weight gain, enhance weight loss, decrease or prevent insulin resistance or enhance metabolism.

Cell permeable inhibitory peptides to SHIP2, e.g., a cell permeable peptide that binds the SH2 domain of SHIP2, may also be used in the thereapeutic method of the invention. The preparation of cell permeable peptides is known to the art, see for example, Chico et al. (2003) Peptides 24:3-9, which reference is herein specifically incorporated by reference in its entirety.

Inhibitory Nucleic Acids

In specific embodiments, SHIP2 gene expression is inhibited with nucleic acid molecules capable of interfering with or silencing SHIP2 gene expression. In one embodiment, SHIP2 expression is inhibited by SHIP2 antisense nucleic acid comprises at least 6 to 200 nucleotides that are antisense to a gene or cDNA encoding SHIP2 or a portion thereof. As used herein, a SHIP2 “antisense” nucleic acid refers to a nucleic acid capable of hybridizing by virtue of some sequence complementarity to a portion of an RNA (preferably mRNA) encoding SHIP2. The antisense nucleic acid may be complementary to a coding and/or noncoding region of an mRNA encoding SHIP2. The oligonucleotides can be DNA or RNA or chimeric mixtures or derivatives or modified versions thereof, can be single- or double-stranded, and can be modified at the base moiety, sugar moiety, or phosphate backbone. The oligonucleotide may include other appended groups such as peptides; agents that facilitate transport across the cell membrane (see, e.g., Letsinger et al. (1989) Proc. Natl. Acad. Sci. USA 86:6553-6556) or blood-brain barrier (see, e.g WO 89/10134,). Such antisense nucleic acids have utility as compounds that inhibit SHIP2 expression, and can be used in the treatment of obesity. Such molecules are known to the art, e.g., for example, as described in U.S. Pat. No. 6,025,198 (Bennett et al.), which publication is herein specifically incorporated by reference in its entirety.

In another embodiment, SHIP2 may be inhibited with ribozymes or triple helix molecules which decrease SHIP2 gene expression. Ribozyme molecules designed to catalytically cleave gene mRNA transcripts encoding SHIP2 can be used to prevent translation of SHIP2 mRNA and, therefore, expression of the gene product. (See, e.g., PCT International Publication WO90/11364). Alternatively, the endogenous expression of SHIP2 can be reduced by targeting deoxyribonucleotide sequences complementary to the regulatory region of the gene (i.e., the gene promoter and/or enhancers) to form triple helical structures that prevent transcription of SHIP2 in target cells in the body (see, for example, Helene et al. (1992) Ann. N.Y. Acad. Sci., 660, 27-36).

In another embodiment, SHIP2 is inhibited by a short interfering RNA (siRNA) through RNA interference (RNAi) or post-transcriptional gene silencing (PTGS) (see, for example, Ketting et al. (2001) Genes Develop. 15:2654-2659). siRNA molecules can target homologous mRNA molecules for destruction by cleaving the mRNA molecule within the region spanned by the siRNA molecule. Accordingly, siRNAs capable of targeting and cleaving homologous SHIP2 mRNA are useful for treating obesity.

Methods of Administration

The invention provides methods of treatment comprising administering to a subject an effective amount of an agent capable of inhibiting SHIP2 activity or gene expression. In a preferred aspect, the agent is substantially purified (e.g., substantially free from substances that limit its effect or produce undesired side-effects). The subject is preferably an animal, e.g., such as cows, pigs, horses, chickens, cats, dogs, etc., and is preferably a mammal, and most preferably human.

Various delivery systems are known and can be used to administer an agent of the invention, e.g., encapsulation in liposomes, microparticles, microcapsules, recombinant cells capable of expressing the compound, receptor-mediated endocytosis (see, e.g., Wu and Wu (1987) J. Biol. Chem. 262:4429-4432), construction of a nucleic acid as part of a retroviral or other vector, etc. Methods of introduction can be enteral or parenteral and include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved, for example, and not by way of limitation, by local infusion during surgery, topical application, e.g., by injection, by means of a catheter, or by means of an implant, said implant being of a porous, non-porous, or gelatinous material, including membranes, such as sialastic membranes, fibers, or commercial skin substitutes.

In another embodiment, the active agent can be delivered in a vesicle, in particular a liposome (see Langer (1990) Science 249:1527-1533). In yet another embodiment, the active agent can be delivered in a controlled release system. In one embodiment, a pump may be used (see Langer (1990) supra). In another embodiment, polymeric materials can be used (see Howard et al. (1989) J. Neurosurg. 71:105). In another embodiment where the active agent of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see, for example, U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression, by homologous recombination.

Pharmaceutical Compositions

The present invention also provides pharmaceutical compositions comprising a therapeutically effective amount of an agent capable of inhibiting SHIP2 activity or gene expression, and a pharmaceutically acceptable carrier. In specific embodiments, the agent is a compound identified by the in vivo screening method of the invention, a SHIP2 antisense molecule, a siRNA molecule, or an antibody to SHIP2. In a more specific embodiment, the composition comprises a combination of an agent of the invention and a second agent. The tern “pharmaceutically acceptable” means approved by a regulatory agency of the Federal or a state government or listed in the U.S. Pharmacopeia or other generally recognized pharmacopoeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin.

In a preferred embodiment, the composition is formulated in accordance with routine procedures as a pharmaceutical composition adapted for intravenous administration to human beings. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The active agents of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the active agent of the invention which will be effective in the treatment of a SHIP2-mediated condition can be determined by standard clinical techniques based on the present description. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. However, suitable dosage ranges for intravenous administration are generally about 20-500 micrograms of active compound per kilogram body weight. Suitable dosage ranges for intranasal administration are generally about 0.01 μg/kg body weight to 1 mg/kg body weight. Effective doses may be extrapolated from dose-response curves derived from in vitro or animal model test systems.

Kits

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

Transgenic Animals

The invention includes a knock-out or knock-in animal having a modified endogenous SHIP2 gene and/or expressing a human SHIP2 gene. The invention contemplates a transgenic animal having an exogenous SHIP2 gene generated by introduction of any SHIP2-encoding nucleotide sequence which can be introduced as a transgene into the genome of a non-human animal. Any of the regulatory or other sequences useful in expression vectors can form part of the transgenic sequence. A tissue-specific regulatory sequence(s) can be operably linked to the transgene to direct expression of the SHIP2 protein to particular cells.

Knock-out animals containing a modified SHIP2 gene as described herein are useful to identify SHIP2 function. Methods for generating knock-out or knock-in animals by homologous recombination in ES cells are known to the art. Animals generated from ES cells by microinjection of ES cells into donor blastocytes to create a chimeric animal, which chimeric animal can be bred to produce an animal in which every cell contains the targeted modification. A transgenic animal can be produced by introducing nucleic acid into the male pronuclei of a fertilized oocyte, e.g., by microinjection, retroviral infection, and allowing the oocyte to develop in a pseudopregnant female foster animal.

Transgenic knock-in animals containing an exogenous human SHIP2 gene are useful in an in vivo screening method for identifying agents capable of inhibiting SHIP2. Such agents are potential therapeutics in the treatment of human SHIP2-mediated conditions, such as obesity and/or insulin resistance. An in vivo screening model is particularly useful because various physiological factors that are present in vivo and that could effect activator binding, SHIP2 activation, and signal transduction, may not be evident from in vitro cell-free or cell-based assays.

Specific Embodiments

The prior art, which demonstrated that genetic deletion of both alleles for SHIP2 resulted in perinatal death due to severe hypoglycemia and the subsequent characterization of SHIP2 heterozygous mice, suggested the presence of increased insulin sensitivity in vivo. However, the prior art SHIP2 knockout mice had the first 18 exons left intact, thus it is unlikely that such animals were genetically null because it is expected that a portion of the protein was still being made and functioned as a dominant negative molecule. Such a model would suggest that the phenotype observed (increased insulin sensitivity) is the result of a dominant negative action on both SHIP1 and SHIP2 actions.

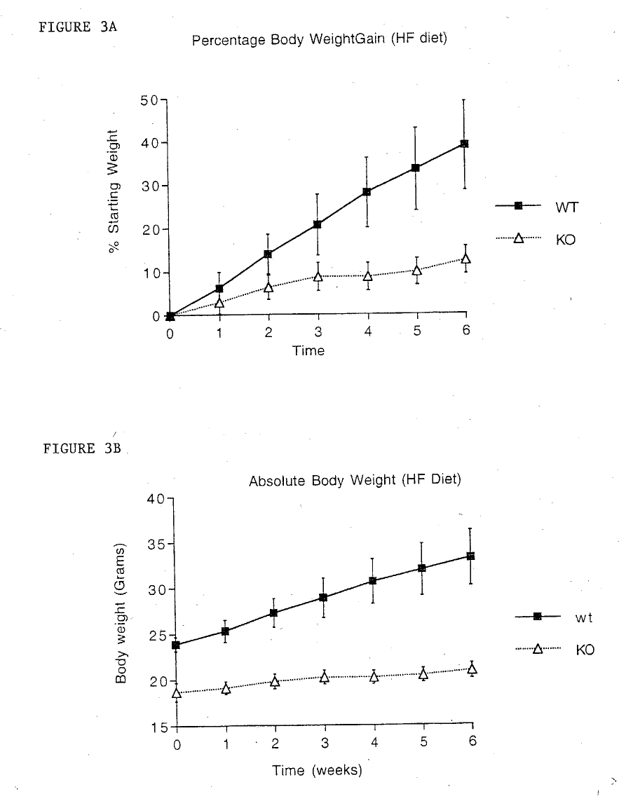

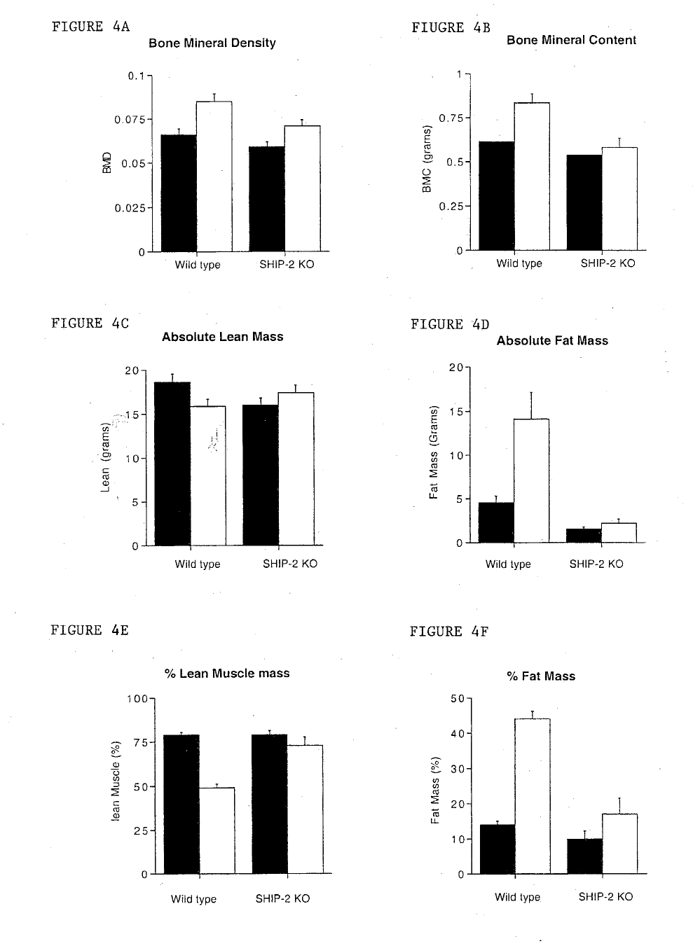

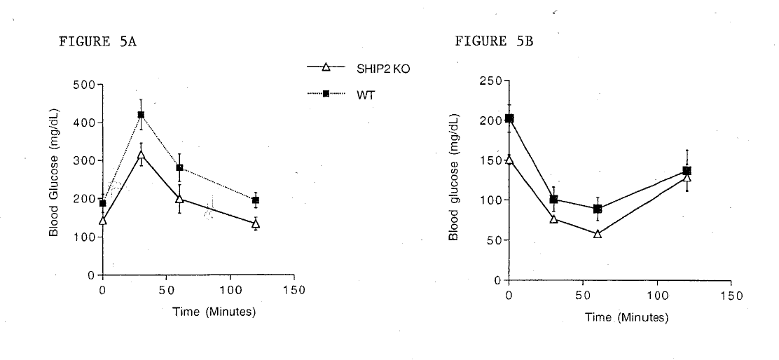

To clarify the role of SHIP2, the SHIP2 null mice were generated, as described below, by deletion of the coding region between the ATG and exon 18. The results demonstrate that SHIP2 deficient mice exhibited an altered phenotype, relative to wild-type animals, in response to a high caloric diet. SHIP2^{-/-} mice, which initially have lower body weight than wild-type, only gained an average of 10% on a high fat diet in comparison to a 45% gain in weight for wild-type littermates (FIGS. 3A and B). This analysis would at first suggest that lean muscle mass is significant reduced in the SHIP2^{-/-} mice, however, when expressed as a percentage of body weight, it represents a high proportion of the total mass than in the wild-type control mice. Intriguingly, this tissue mass, which actually has the greatest expression of SHIP2, enjoys a significant degree of sparing in response to a high diet (FIGS. 4E-F). The lack of weight gain even when these mice are given a high caloric diet can be attributed to the lack of deposition of adipose tissue as highlighted by the absolute and percentage fat mass for the SHIP2^{-/-} mice (FIGS. 4B-C). These SHIP2^{-/-} mice fail to develop the hyperglycemia or show an impaired glucose tolerance typically associated with a high fat diet. Similarly, they have a near equivalent response in blood glucose to exogenous insulin before the high fat diet challenge (FIGS. 5A-B) and show an equal ability to phosphorylate downstream signaling substrate such as Akt and p70s6k kinase.

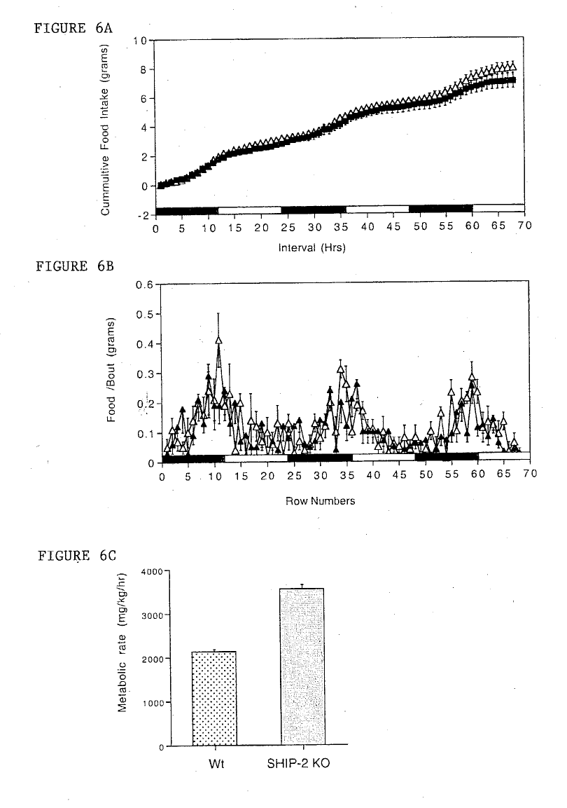

One possible energy dissipating mechanism is the mitochondrial uncoupling of ATP-synthesis from respiratory chain oxidation through the uncoupling protein UCP-1, whereby the energy derived from food could be dissipated as heat instead of stored as ATP. The fact that these animals are able to maintain a normal body weight regulation during an episode of excessive caloric intake (high fat diet) would suggest that a greater than normal activation of thermogenic response in tissues such as brown adipose depot. Indirect calorimetric analysis of SHIP2^{-/-} and wild-type mice showed that SHIP2^{-/-} mice had an increased basal metabolic rate (2432±17 wt c.f. to 3189±291; ANOVA P>0.05) (FIG. 6) as well a change in CO₂ production, and derived energy expenditure despite have equal food intake during this period.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for.

### Example 1

### Expression of Human SHIP2

Knock-out mice containing a lacZ gene insertion into the endogenous SHIP2 locus were generated using the technology described in co-pending U.S. Ser. No. 10/076,840 filed 15 Feb. 2002 (US Patent Application Publication 20020106628), herein specifically incorporated by reference in its entirety. No change was observed in the expected birth ratio for F2 homozygous (SHIP2^{/}), heterozygous (SHIP2^{-/+}) and wild-type (SHIP2^{+/+}) mice as predicted by Mendellian genetics and all progeny reached normal developmental milestones through the first 6 weeks of age (data not shown). SHIP2^{-/-} deficient mice exhibited a significantly reduction in bodyweight through the first 8 weeks of development and a distinctive facial abnormality; however this was not associated with a change in normal daily food intake through the 18 weeks of assessment period.

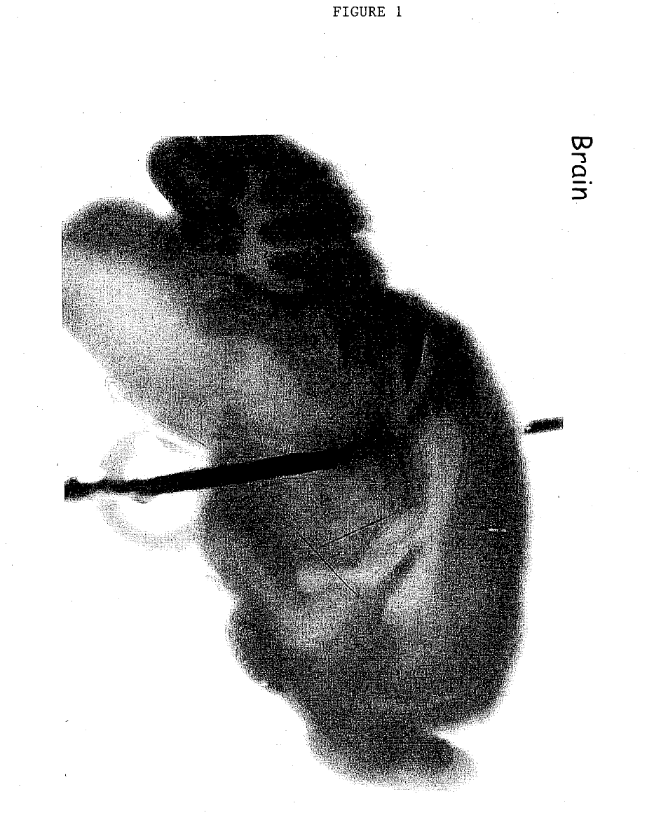

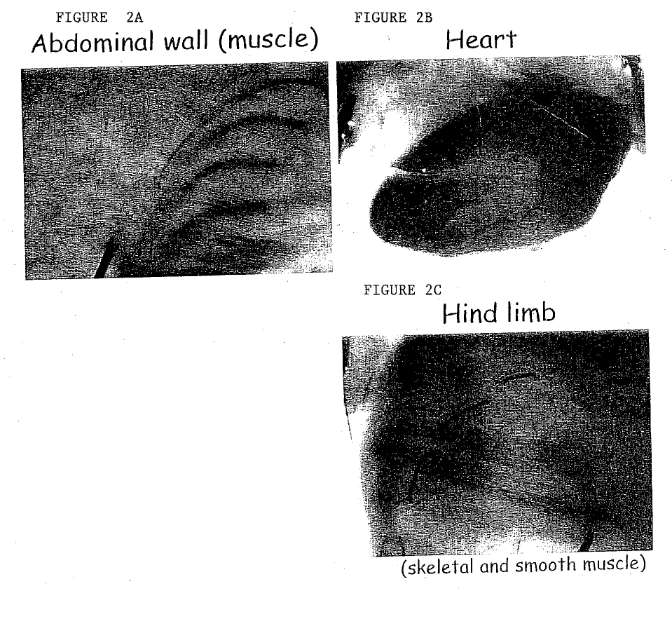

Extensive Lac-Z expression could be detected throughout the brain (FIG. 1) as well as in all muscle tissues type such as the abdominal wall (smooth), heart (cardiac), and hindlimb (skeletal) muscle (FIGS. 2A, 2B, and 2C).

### Example 2

### Analysis of Metabolic Parameters of SHIP-2^{-/-} Knockout Mice

Male mice were obtained at 7-8 weeks of age, and were housed in rooms with 12 hours of light per day at 69-74° C. and 40-60% humidity. All experiments began at 8-10 weeks of age. Metabolic measurements were obtained using an Oxymax (Columbus Instruments International Corp., Columbus, Ohio) open circuit indirect calorimetry system. The system was calibrated against standard gas mixture to measure O₂ consumed (ml/kg/hr) and CO₂ generated (ml/kg/hr) by each animal at 57 min intervals for a 23-24 hr period. Energy expenditure (or heat) was calculated as the product of calorific value of oxygen (=3.815±1.232 x respiratory quotient) and the volume of O₂ consumed. The first 2 h of measurements was used as a period of adaptation for the animals and metabolic rate and activity were evaluated for a 24 hr period. Serum samples reported were taken between 10:00 and 12:00 hours and analyzed for a 10 points chemistry of glucose, triglycerides and cholesterol utilizing the Bayer 1650 blood chemistry analyzer (Bayer, Tarrytown N.Y.). Non-esterified free fatty acid's (NEFA) were analyzed by a diagnostic kit (WAKO, Richmond, Va.) and insulin levels by ELISA (Linco, St. Charles, Mo.).

Both SHIP2^{-/-} and littermate control mice exhibited similar fasting glucose levels, insulin profiles, ability to dispose of an oral bolus of glucose, and non-fasting glucose levels. Consistent with these results, SHIP2^{-/-} mice had equivalent basal metabolic rate (SHIP2 mean VO₂, 2550±252 wt to 2537±239, NS) approximately equal respiratory quotient ratios and energy expenditure relative to control littermates, as determined by indirect calorimetry.

### Example 3

### Altered Phenotype of SHIP2′-Knockout Mice to a High Fat Diet

Wild-type and SHIP2^{-/-} mice of 8-10 weeks of age were maintained on normal chow or 45% high fat diet (Research Diets, N.J.) for 6 weeks and their bodyweights measured at the same time for 6 weeks. Body composition was further analyzed by dual emission X ray absorption (DEXA) analysis before and after high fat (HF) diet challenge (FIGS. 3A-B). Both groups were acclimated for a 24 hr period in metabolic cage and ad lib food intake, O₂ consumption, CO₂ production and activity continuously monitored for the next 48 hr by indirect calorimetry. The results are shown in FIG. 6.

What is claimed is:

1. A non-human transgenic animal comprising an alteration of the endogenous SH2 domain containing inositol 5-phosphatase (SHIP2) gene.

2. The transgenic animal of claim 1, wherein the transgenic animal is a knockout animal comprising a disrupted SHIP2 gene.

3. The transgenic animal of claim 2, wherein the transgenic animal is homozygous for the disrupted SHIP2 gene and exhibits resistance to development of obesity when placed on a high fat diet.

4. The transgenic animal of claim 1, wherein the transgenic animal is a knock-in animal comprising a human SHIP2 gene.

5. The transgenic animal of claim 3, wherein the transgenic animal is further comprising a human SHIP2 gene.

6. The transgenic knockout animal of claim 1, wherein the animal is selected from the group consisting of mice, rats, rabbits, monkeys, guinea pigs, dogs and cats.

7. An in vivo screening method for identifying agents capable of inhibiting SHIP2 activity or gene expression, comprising:
(a) administering a test agent to the animal of claim 4; and
(b) determining the ability of the test agent to inhibit SHIP2 activity or gene expression.

8. The screening method of claim 7, wherein the ability of a test agent to inhibit SHIP2 activity or gene expression is determined by comparing weight gain in response to a high fat diet relative to a control wild-type animal, wherein resistance to weight gain relative to the control wild-type animal indicates inhibition of SHIP2 activity or gene expression.

9. The screening method of claim 7, wherein the ability of a test agent to inhibit SHIP2 activity or gene expression is determined by comparing glucose tolerance in response to a high fat diet relative to a wild-type control animal, wherein an improved glucose tolerance relative to the wild-type control animal indicates inhibition of SHIP2 activity or gene expression.

10. The screening method of claim 7, wherein the ability of a test agent to inhibit SHIP2 activity or gene expression is determined by comparing basal metabolic rate in response to a high fat diet relative to a wild-type control animal, wherein an increased basal metabolic rate relative to the wild-type control animal indicates inhibition of SHIP2 activity or gene expression.

11. The screening method of claim 7, wherein the ability of a test agent to inhibit SHIP2 gene expression is determined by obtaining a biological sample from a test animal, and measuring SHIP2 gene expression.

12. The screening method of claim 11, wherein SHIP2 gene expression is determined by measuring mRNA corresponding to SHIP2, wherein a decreased amount of SHIP2 mRNA relative to a control indicates a test agent capable of inhibiting SHIP2 gene expression.

13. The screening method of claim 11, wherein SHIP2 activity is determined by measuring Akt activation, wherein increased Akt activation relative to a control indicates a test agent capable of inhibiting SHIP2 activity.

14. The screening method of claim 11, wherein the biological sample is fluid, cells, or tissue.

15. A therapeutic method for treating obesity, comprising administering a therapeutically effective amount of an agent capable of inhibiting SHIP2-mediated activity.

16. The therapeutic method of claim 15, wherein the agent is identified by the screening method of claim 6.

17. The therapeutic method of claim 15, wherein obesity results from weight gain associated with a high fat diet.

18. The method of claim 15, wherein the agent is an inhibitor SHIP2 activity or gene expression.

19. The method of claim 18, wherein the agent is an inhibitor of SHIP2 gene expression and is an SHIP2 antisense molecule.

20. The method of claim 18, wherein the agent is an antibody specific for SHIP2.
